(19) United States
(12) Patent Application Publication
(10) Pub. No.: US 2005/0158314 A1
(43) Pub. Date: Jul. 21, 2005

(54) Method of therapeutic administration of anti-CD40L compounds

(76) Inventors: Susan L. Kalled, Jamaica Plain, MA (US); David W. Thomas, Wellesley, MA (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 11/021,599

(22) Filed: Dec. 23, 2004

Related U.S. Application Data
(63) Continuation of application No. 10/015,832, filed on Dec. 12, 2001, now abandoned.
Application No. 10/015,832 is a continuation of application No. 09/346,653, filed on Jul. 1, 1999, now abandoned.
Application No. 09/346,653 is a continuation of application No. PCT/US98/00573, filed on Jan. 9, 1998.
(60) Provisional application No. 60/034,672, filed on Jan. 10, 1997.
(60) Provisional application No. 60/040,154, filed on Mar. 7, 1997.

(51) Int. Cl. A61K 39/395

(52) U.S. Cl. 424/144.1

(57) ABSTRACT

This invention relates to regimens for therapeutically administering anti-CD40L compounds to patients. Immune-related disorders can be effectively treated by administering anti-CD40L compounds at intervals of three weeks or more.

### FIELD OF THE INVENTION

The invention relates to regimens for therapeutically administering anti-CD40L compounds to patients.

### BACKGROUND OF THE INVENTION

One of the necessary reactions in the generation of antibodies is the interaction of CD40 on B cells with CD40 ligand (CD40L) on activated T cells, a step which is required for B cell growth and subsequent production of antibodies. (Note: “gp39” is used synonymously for CD40L in some reports.) As further described below, a number of anti-CD40L compounds have been produced, and some have been tested in animals for efficacy in altering the course of antibody-associated diseases.

The protocols used in the reported experiments on effects of anti-CD40L compounds on animals with immune disorders have employed doses of the compounds administered to the animals at intervals of two weeks or less, with typical intervals between treatments being 1-7 days. (See, e.g., Mohan et al., J. Immunol. 154: 1470-1480, 1995; Early et al., J. Immunol. 157: 3159-3164, 1996; Stüber et al., J. Exp. Med. 183:693-698, 1996; Chen et al., J. Immunol. 155:2833-2840, 1995; Gerritse et al., Proc. Nat. Acad. Sci. 93:2499-2504, 1996; Green et al., T. Virol. 70:2569-2575, 1996; Durie et al., Science 261:1328-1330, 1993; Durie et al., J. Clin. Invest. 94:1333-1338, 1994; Larsen et al., Transplantation 61:4-9, 1996; and Griggs et al., J. Exp. Med 183:801-810, 1996). There has been no available information that suggests that less frequent administration of anti-CD40L compounds would be efficacious in inhibiting the production of pathologic antibodies or improving the course of immune-related diseases.

### SUMMARY OF THE INVENTION

The inventors have demonstrated that administering an anti-CD40L compound at intervals of three weeks or more is effective in treatment of disorders with antibody-related pathogenesis. The invention provides a method of treating a patient with an antibody-related disease, which includes administering a therapeutically effective amount of an anti-CD40L compound to the patient on a first day and again on a second day, with at least about 3 weeks between the first day and the second day. Similar amounts of an anti-CD40L compound may subsequently be given to the patient, with at least about 3 weeks between successive doses. In one embodiment, the interval between doses is at least about 4 weeks.

In another aspect of the invention, a method is taught for treating a patient with an antibody-related disease, comprising administering a therapeutically effective amount of an anti-CD40L compound to the patient for a first therapeutic period at intervals of less than about 3 weeks, then administering a therapeutically effective amount of an anti-CD40L compound to the patient for a second therapeutic period at intervals of at least about 3 weeks or at least about 4 weeks.

A further application for the above-described administration regimens is for treating a patient with a chronic immune system disorder, such as psoriasis, allergic conditions, arthritis or multiple sclerosis.

In another embodiment, the methods of the invention are useful for treating a chronic autoimmune disease, such as systemic lupus erythematosis, myasthenia gravis, autoimmune hemolytic anemia, idiopathic thrombocytopenic purpura, or anti-phospholipid syndrome.

Yet another aspect of the invention provides a method of inhibiting rejection of transplanted tissue within a patient; this method includes administering a therapeutically effective amount of an anti-CD40L compound to the patient on a first day and again on a second day, with at least about 3 weeks between the first day and the second day. The compound may be given subsequently at varying intervals, but in one embodiment, it is given subsequent to the second dose at intervals of at least about 3 weeks or at least about 4 weeks. The transplanted tissue may be any organ or tissue which is suitable for transplantation. Particularly intended for inclusion are transplants of skin, kidney, liver, heart, bone marrow, or eye tissue. The graft may be an allograft or a xenograft.

The methods of the invention may also be useful in suppressing immune reaction after gene therapy.

In an alternative dosing regimen for inhibiting rejection of transplanted tissue within a patient, an anti-CD40L compound is administered to the patient for a first therapeutic period at intervals of less than about 3 weeks, then administering for a second therapeutic period at intervals of at least about 3 weeks.

The anti-CD40L compound may be any compound that binds to CD40L on the surface of CD40L-expressing cells, such as activated T cells. In one embodiment, the compound is an anti-CD40L antibody, preferably a monoclonal antibody. The monoclonal antibody may be 5c8 (ATCC Accession No. HB 10916).

The anti-CD40L compound may be formulated in a therapeutic composition which includes a therapeutically-effective amount of the anti-CD40L compound and a pharmaceutically acceptable carrier. The therapeutic composition may also include a second therapeutically effective compound.

### BRIEF DESCRIPTION OF THE DRAWINGS

FIG. 1 is a chart of the changes with time in several measured characteristics of blood and urine from control and treated (SWR X NZB) F₁ mice in Experiment II. The anti-CD40L mAb MR1, at 500 ug/animal i.p., was administered once when the mice were 4 months old, again at 7 months of age, again at 9 months, and then at monthly intervals. Each of the upper five rows of the chart, marked AR-BN, contains data from a single control animal, and each of the lower six rows, marked CL-CR, contains data from a single treated animal. This study began when the animals were 4 months of age, in February 1996. The vertical double lines separate 4 groups of data, each data group providing the measurements for urine and blood samples collected on the date listed above the data Proteinuria (PU) levels are indicated from trace to level 4. Level 1 correlates with urine albumin of 30 mg/dl albumin, level 2 with 100 mg/dl, level 3 with 300 mg/dl, and level 4 with over 2000 mg/dl. Levels of anti-MR1 antibodies (provided in column labeled “anti-MR1”), anti-ssDNA antibodies and anti-dsDNA antibodies are given in μg/ml blood. Where appropriate, values are given as mean and standard deviation of several samples, in the form mean(S.D.). A dash indicates that a sample was not collected, typically because the animal had died. ND refers to “not done.”

FIG. 2 is a chart of proteinuria measurements of the Experiment II animals over time. The first column provides the animal numbers as in FIG. 1. The columns are headed with the dates of sample collection. NC means “not collected.”

FIG. 3 is a chart of blood and urine characteristics with time in Experiment V control and untreated mice, which started treatment at 4.5 months of age. MR1 was administered to treated animals once at 500 ug/animal i.p. when the mice were 4.5 months old, and then as monthly injections of 500 ug, i.p. Each of the upper seven rows of the chart, marked AR-BLR, contains data from a single control animal, and each of the lower seven rows, marked CR-CLR, contains data from a single treated animal. This study began when the animals were 4.5 months of age, in May 1996. Other descriptions of the figure are the same as those of FIG. 1.

FIG. 4 is a chart of proteinuria measurements of the Experiment V animals over time. Animal numbers are as described for FIG. 3. Other descriptions of the figure are the same as those of FIG. 2.

FIG. 5 is a chart of chart of blood and urine characteristics with time in Experiment VII control and untreated mice, which started treatment at 5.5 months of age. MR1 was administered to treated animals once weekly at 500 ug/animal i.p. for six weeks, followed by monthly injections of 500 ug, i.p. Each of the upper three rows of the chart, marked AN-BL, contains data from a single control animal (as noted in FIG. 6, some control animals had died before the data for FIG. 5 was collected), and each of the lower seven rows, marked CR-DN, contains data from a single treated animal. This study began when the animals were 5.5 months of age, in June 1996. Other descriptions of the figure are the same as those of FIG. 1.

FIG. 6 is a chart of proteinuria measurements of the Experiment VII animals over time. Each of the upper seven rows of the chart, marked AR-BN, contains data from a single control animal, and each of the lower seven rows, marked CR-DN, contains data from a single treated animal. Other descriptions of the figure are the same as those of FIG. 2.

FIG. 7 is a chart of blood and urine characteristics with time in Experiment X control and untreated mice, which started treatment at 5.5 months of age. MR1 was administered to treated animals once weekly at 500 ug/animal i.p. for four weeks, followed by monthly injections of 200 ug, i.p. Each of the upper eight rows of the chart, marked AR-BE, contains data from a single control animal, and each of the lower eight rows, marked CR-DLR, contains data from a single treated animal. This study began when the animals were 5.5 months of age, in October 1996. Other descriptions of the figure are the same as those of FIG. 1.

FIG. 8 is a chart of proteinuria measurements of the Experiment X animals over time. The first column provides the animal numbers as in FIG. 7. Other descriptions of the figure are the same as those of FIG. 2.

FIG. 9 is a chart of blood and urine characteristics with time in Experiment VI control and untreated mice, which started treatment at 7 months of age. MR1 was administered to 4 treated animals once weekly at 500 ug/animal i.p. for six weeks, followed by monthly injections of 500 ug, i.p. Each of the lower four rows, marked DN-EN, contains data from a single treated animal. At the time of first data collection for this chart, all control animals had died, as noted FIG. 10. This study began when the animals were 7 months of age, in June 1996. Other descriptions of the figure are the same as those of FIG. 1.

FIG. 10 is a chart of proteinuria measurements of the Experiment VI animals over time. Each of the upper four rows of the chart, marked AR-CN, contains data from a single control animal, and each of the lower four rows, marked DN-EN, contains data from a single treated animal. Other descriptions of the figure are the same as those of FIG. 2.

### DETAILED DESCRIPTION OF THE INVENTION

The method of the invention involves treating, preventing, reversing or stabilizing a patient with an antibody-related disease, by treating the patient with an anti-CD40L compound at intervals of more than two weeks. The compound blocks the interaction of CD40L on T cells with CD40 on B cells, which is thought to inhibit the production of pathologic antibodies responsible for many of the pathologic effects of various autoimmune diseases and chronic immune disorders.

Compounds

Therapeutic compounds useful for the methods of the invention include any compound that blocks the interaction of CD40 on B cells with CD40L expressed on the surface of activated T cells. Anti-CD40L compounds specifically contemplated include polyclonal antibodies and monoclonal antibodies (mAbs), as well as antibody derivatives such as chimeric molecules, humanized molecules, molecules with reduced effector functions, bispecific molecules, and conjugates of antibodies. In a preferred embodiment, the antibody is 5c8, as described in U.S. Pat. No. 5,474,771, the specification of which is hereby incorporated by reference. Other known antibodies against 5c8 antigen include antibodies ImxM90, ImxM91 and ImxM92 (obtained from Immunex), an anti-CD40L mAb commercially available from Ancell (clone 24-31, catalog #353-020, Bayport, Minn.), and an anti-CD40L mAb commercially available from Genzyme (Cambridge, Mass., catalog #80-3703-01). Also commercially available is an anti-CD40L mAb from PharMingen (San Diego, catalog #33580D). Numerous additional anti-CD40L antibodies have been produced and characterized (see, e.g., WO 96/23071 of Bristol-Myers Squibb, the specification of which is hereby incorporated by reference).

The invention also includes anti-CD40L molecules of other types, such as complete Fab fragments, F(ab′)₂ compounds, V_{H} regions, F_{V} regions, single chain antibodies (see, e.g., WO 96,23071), polypeptides, fusion constructs of polypeptides, fusions of CD40 (such as CD40Ig, as in Hollenbaugh et al., J. Immunol. Meth. 188:1-7, 1995, which is hereby incorporated by reference), and small molecule compounds such as small semi-peptidic compounds or non-peptide compounds, all capable of blocking the CD40-CD40L interaction. Procedures for designing, screening and optimizing small molecules are provided in the patent application PCT/US96/10664, filed Jun. 21, 1996, the specification of which is hereby incorporated by reference.

Various forms of antibodies may also be produced using standard recombinant DNA techniques (Winter and Milstein, Nature 349: 293-99, 1991). For example, “chimeric” antibodies may be constructed, in which the antigen binding domain from an animal antibody is linked to a human constant domain (an antibody derived initially from a nonhuman mammal in which recombinant DNA technology has been used to replace all or part of the hinge and constant regions of the heavy chain and/or the constant region of the light chain, with corresponding regions from a human immunoglobin light chain or heavy chain) (see, e.g., Cabilly et al., U.S. Pat. No. 4,816,567; Morrison et al., Proc. Natl. Acad. Sci. 81: 6851-55, 1984). Chimeric antibodies reduce the immunogenic responses elicited by animal antibodies when used in human clinical treatments.

In addition, recombinant “humanized” antibodies may be synthesized. Humanized antibodies are antibodies initially derived from a nonhuman mammal in which recombinant DNA technology has been used to substitute some or all of the amino acids not required for antigen binding with amino acids from corresponding regions of a human immunoglobin light or heavy chain (chimeras comprising mostly human IgG sequences into which the regions responsible for specific antigen-binding have been inserted)(see, e.g., PCT patent application WO 94/04679). Animals are immunized with the desired antigen, the corresponding antibodies are isolated and the portion of the variable region sequences responsible for specific antigen binding are removed. The animal-derived antigen binding regions are then cloned into the appropriate position of the human antibody genes in which the antigen binding regions have been deleted. Humanized antibodies minimize the use of heterologous (inter-species) sequences in human antibodies and are less likely to elicit immune responses in the treated subject.

Also useful in the methods and compositions of this invention are primate or primatized antibodies.

Antibody fragments and univalent antibodies may also be used in the methods and compositions of this invention. Univalent antibodies comprise a heavy chain/light chain dimer bound to the Fc (or stem) region of a second heavy chain. “Fab region” refers to those portions of the chains which are roughly equivalent, or analogous, to the sequences which comprise the Y branch portions of the heavy chain and to the light chain in its entirety, and which collectively (in aggregates) have been shown to exhibit antibody activity. A Fab protein includes aggregates of one heavy and one light chain (commonly known as Fab′), as well as tetramers which correspond to the two branch segments of the antibody Y, (commonly known as F(ab)₂), whether any of the above are covalently or non-covalently aggregated, so long as the aggregation is capable of selectively reacting with a particular antigen or antigen family.

In addition, standard recombinant DNA techniques can be used to alter the binding affinities of recombinant antibodies with their antigens by altering amino acid residues in the vicinity of the antigen binding sites. The antigen binding affinity of a humanized antibody may be increased by mutagenesis based on molecular modeling (Queen et al., Proc. Natl. Acad. Sci. 86:10029-33, 1989; PCT patent application WO 94/04679). It may be desirable to increase or to decrease the affinity of the antibodies for CD40L, depending on the targeted tissue type or the particular treatment schedule envisioned. This may be done utilizing phage display technology (see, e.g., Winter et al., Ann. Rev. Immunol. 12:433-455, 1994; and Schier et al., J. Mol. Biol. 255:28-43, 1996, which are hereby incorporated by reference). For example, it may be advantageous to treat a patient with constant levels of antibodies with reduced affinity for CD40L for semi-prophylactic treatments. Likewise, antibodies with increased affinity for CD40L may be advantageous for short-term treatments.

Subjects

The term “patient” is taken to mean any mammalian patient to which anti-CD40L compounds may be administered. Patients specifically intended for treatment with the method of the invention include humans, as well as nonhuman primates, sheep, horses, cattle, goats, pigs, dogs, cats, rabbits, guinea pigs, hamsters, gerbils, rats and mice, as well as the organs, tumors, and cells derived or originating from these hosts.

The subjects for which the methods of the invention are intended have disease related to antibody production.

Routes of Administration

The compounds of the invention may be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the invention, by such means as depot injections. Some forms of anti-CD40L compounds may be suitable for oral administration, and could be formulated as suspensions or pills.

Dosages and Frequency of Treatment

The amount of and frequency of dosing for any particular compound to be administered to a patient for a given immune complex disease is a judgment made by the patient's physician, based on a number of factors. The general dosage is established by preclinical and clinical trials, which involve extensive experiments to determine the beneficial and deleterious effects on the patient of different dosages of the compound. Even after such recommendations are made, the physician will often vary these dosages for different patients based on a variety of considerations, such as a patient's age, medical status, weight, sex, and concurrent treatment with other pharmaceuticals. Determining the optimal dosage for each anti-CD40L compound used to treat lupus nephritis is a routine matter for those of skill in the pharmaceutical and medical arts.

Various regimens may be used for treatment of lupus or other immune complex diseases according to this invention. Generally, the frequency of dosing would be determined by the attending physician, and might include periods of greater dosing frequency, such as at daily or weekly intervals, alternating with periods of less frequent dosing, such as at monthly or longer intervals.

To exemplify dosing considerations for an anti-CD40L compound, the following examples of administration strategies are given for an anti-CD40L mAb. The dosing amounts could easily be adjusted for other types of anti-CD40L compounds. In general, single dosages of between about 0.05 and about 50 mg/kg patient body weight are contemplated, with dosages most frequently in the 1-20 mg/kg range. For acute treatment, an effective dose of antibodies ranges from about 1 mg/kg body weight to about 20 mg/kg body weight, administered daily for a period of about 1 to 5 days, preferably by bolus intravenous administration. The same dosage and dosing schedule may be used in the load phase of a load-maintenance regimen, with the maintenance phase involving intravenous or intramuscular administration of antibodies in a range of about 0.1 mg/kg body weight to about 20 mg/kg body weight, for a treatment period of anywhere from weekly to 3 month intervals. Chronic treatment may also be carried out by a maintenance regimen, in which antibodies are administered by intravenous or intramuscular route, in a range of about 0.1 mg/kg body weight to about 20 mg/kg body weight, with interdose intervals being anywhere between about 1 week and about to 3 months. In addition, chronic treatment may be effected by an intermittent bolus intravenous regimen, in which between about 1.0 mg/kg body weight and about 100 mg/kg body weight of antibodies are administered, with the interval between successive treatments being from 1 to 6 months. For all except the intermittent bolus regimen, administration may also be by oral, pulmonary, nasal or subcutaneous routes.

Generally, therapy is commenced with low doses of antibodies. For example, an initial dose of antibodies is administered to the patient by, for example, injection or infusion. That initial dose should contain between about 1.0 mg and 30 mg of antibodies per day for a 70 kg patient. For repeated administrations over several days, dosages may be administered on successive days, every two to six days, once a week, every two to four weeks or once a month, until a desired suppression of disease symptoms is observed. However, other dosage regimens are also useful. When the symptoms have been alleviated to the desired level, treatment may cease. Patients may, however, require intermittent treatment on a long term basis upon recurrence of disease symptoms.

According to an alternate embodiment of this invention for treatment of lupus or other antibody-related diseases, the effectiveness of the antibodies may be increased by administration serially or in combination with conventional anti-lupus therapeutic agents or drugs such as, for example, salicylates, corticosteroids or immunosuppressants. Alternatively, the antibodies may be conjugated to a conventional agent. This advantageously permits the administration of the conventional agent in an amount less than the conventional dosage, for example, less than about 50% of the conventional dosage, when the agent is administered as monotherapy. Accordingly, the occurrence of many side effects associated with that agent might be avoided.

Combination therapies according to this invention for treatment of lupus include the use of anti-CD40L antibodies together with agents targeted at B cells, such as anti-CD19, anti-CD28 or anti-CD20 antibody (unconjugated or radiolabeled), IL-14 antagonists, LJP394 (LaJolla Pharmaceuticals receptor blocker), IR-1116 (Takeda small molecule) and anti-Ig idiotype monoclonal antibodies. Alternatively, the combinations may include T cell/B cell targeted agents, such as CTLA4Ig, IL-2 antagonists, IL-4 antagonists, IL-6 antagonists, receptor antagonists, anti-B7 monoclonal antibodies, TNF, LFA1/ICAM antagonists, VLA4/VCAM antagonists, brequinar and IL-2 toxin conjugates (e.g., DAB), prednisone, cyclophosphamide, and other immunosuppressants. Combinations may also include T cell targeted agents, such as CD4 antagonists, CD2 antagonists and IL-12.

Combination therapies for treatment of a patient with a non-lupus immune complex disease might involve administration of an anti-CD40L compound as well as an agent which would typically be administered for the particular immune complex disease in question.

Once improvement of the patient's condition has occuerred, a maintenance dose of anti-CD40L antibodies, alone or in combination with a conventional anti-lupus agent is administered, if necessary. Subsequently, the dosage or the frequency of administration, or both, may be reduced, as a function of the symptoms, to a level at which the improved condition is retained. When the symptoms have been alleviated to the desired level, treatment might cease. In other instances, as determined by a patients physician, occasional treatment might be administered, for example at intervals of four weeks or more. Patients may, however, require intermittent treatment on a long-term basis upon any recurrence of disease symptoms.

Formulation

An anti-CD40L compound used in the methods of the invention is administered in a pharmaceutically-effective amount, which is an amount which produces a medically beneficial effect on a patient with an antibody-related disease, an immune-associated disorder, or a patient with a transplant or a transgene for which suppression of rejection is desirable. Medically beneficial effects would include preventing deterioration or causing improvement in the patient's medical condition. As an example, an organ that is often damaged by pathologic antibodies is the kidney in SLE patients. In these patients, treated with the methods of the invention, renal function and health may be monitored with one or more laboratory tests which measure the concentrations of relevant substances in blood or urine, other urine characteristics, or the rate of clearance of various substances from the blood into the urine. The parameters measured by these tests, either individually or in combination, can be used by a physician to assess renal function or damage. Examples of such parameters include the blood concentration of urea, creatinine or protein; the urine concentration of protein or of various blood cells such as erythrocytes or leucocytes; urine specific gravity; amount of urine; the clearance rates of inulin, creatinine, urea or p-aminohippuric acid; and the presence of hypertension or edema. Medically beneficial effects would also include the diminution of autoantibodies, such as anti-dsDNA antibodies in the serum of lupus patients.

An anti-CD40L compound of the invention is administered to a patient in a pharmaceutically acceptable composition, which may include a pharmaceutically-acceptable carrier. Such a carrier is relatively non-toxic and innocuous to a patient at concentrations consistent with effective activity of the anti-CD40L compound or other active ingredients, so that any side effects ascribable to the carrier do not vitiate the beneficial effects of the active ingredients of the composition. The composition may include other compatible substances; compatible, as used herein, means that the components of the pharmaceutical composition are capable of being commingled with the anti-CD40L compound, and with each other, in a manner such that there is no interaction which would substantially reduce the therapeutic efficacy of the pharmaceutical. Nasal spray formulations comprise purified aqueous solutions of the active compound with preservative agents and isotonic agents. Such formulations are preferably adjusted to a pH and isotonic state compatible with the nasal mucous membranes. Formulations of the present invention suitable for oral administration may be presented as discrete units such as capsules, cachets, tablets, pills or lozenges, each containing a predetermined amount of the potentiating anti-CD40L compound as a powder or granules; as liposomes; or as a suspension in an aqueous liquor or non-aqueous liquid such as a syrup, an elixir, an emulsion or a draught.

Use of Anti-CD40L Compounds Administered at Wide Intervals to Treat Lupus Nephritis in Nonhuman Subjects

We chose to demonstrate the efficacy of administering anti-CD40L compounds in an animal model of lupus nephritis. Systemic lupus erythematosus (SLE) is a life threatening autoimmune disease, characterized by the production of autoantibodies against various tissues, and often against DNA. SLE affects approximately 140,000 people in the United States and 105,000 in western Europe, predominantly women of childbearing age. In most patients, lupus-associated immunoglobulins and immune complexes are deposited in the renal glomeruli, causing a decline in renal function. If widely spaced doses of anti-CD40L compounds are efficacious in selectively suppressing antibody production, such a dosing regime would exert beneficial effects on nephritis. This could be evidenced in treated animals by slower progression of nephritis, reduced severity of nephritis, enhanced survival, or even by improvement of renal function in some animals.

We tested the effects of the hamster anti-muCD40L mAb MR1 on the course of nephritis in the female (SWR X NZB) F₁ mouse, in several studies as described below. Control animals were injected either with Syrian hamster polyclonal Ig or with Ha4/8, an Armenian hamster mAb directed against KLH. Proteinuria levels are indicated from trace to level 4. Level 1 correlates with urine albumin of 30 mg/dl albumin, level 2 with 100 mg/dl, level 3 with 300 mg/dl, and level 4 with over 2000 mg/dl. A level of 2 was considered to indicate moderate nephritis, with 2.5 and greater indicating severe nephritis.

If untreated, or if treated with the nonspecific hamster immunoglobulins administered to control animals, the mice normally die by 12 months of age. While the onset of proteinuria in untreated animals is variable, most have mild to moderate proteinuria by 3 months of age; the proteinuria tends to increase with age. By about 5 months of age, all control animals typically have detectable anti-dsDNA antibodies, and most have detectable anti-ssDNA antibodies; this contrasts with the complete lack of detectable levels of these antibodies in normal mice, such as the female SWR parents of the (SWR X NZB) F₁ mice.

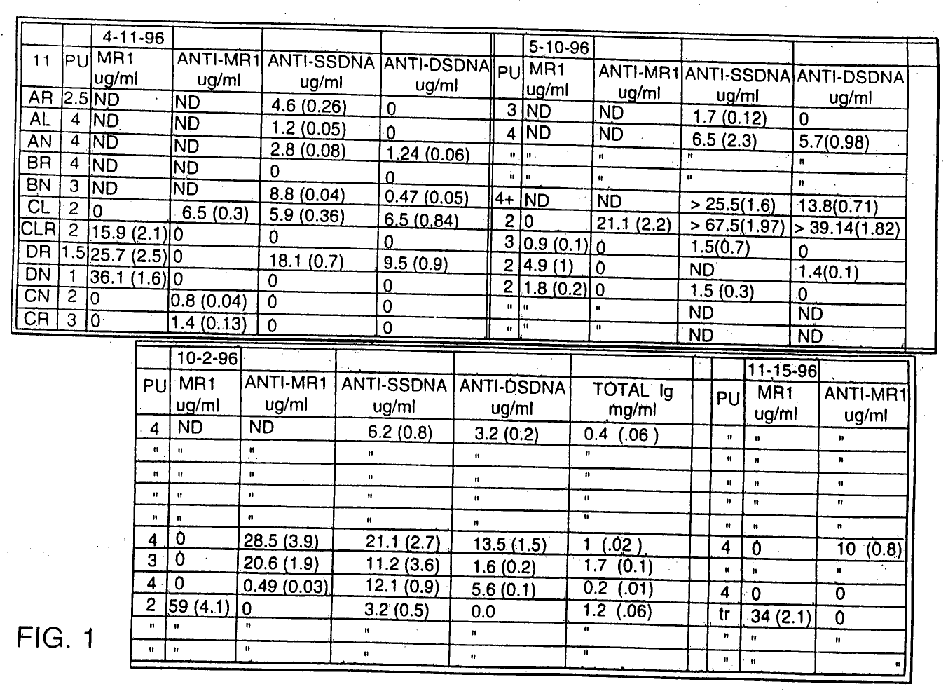

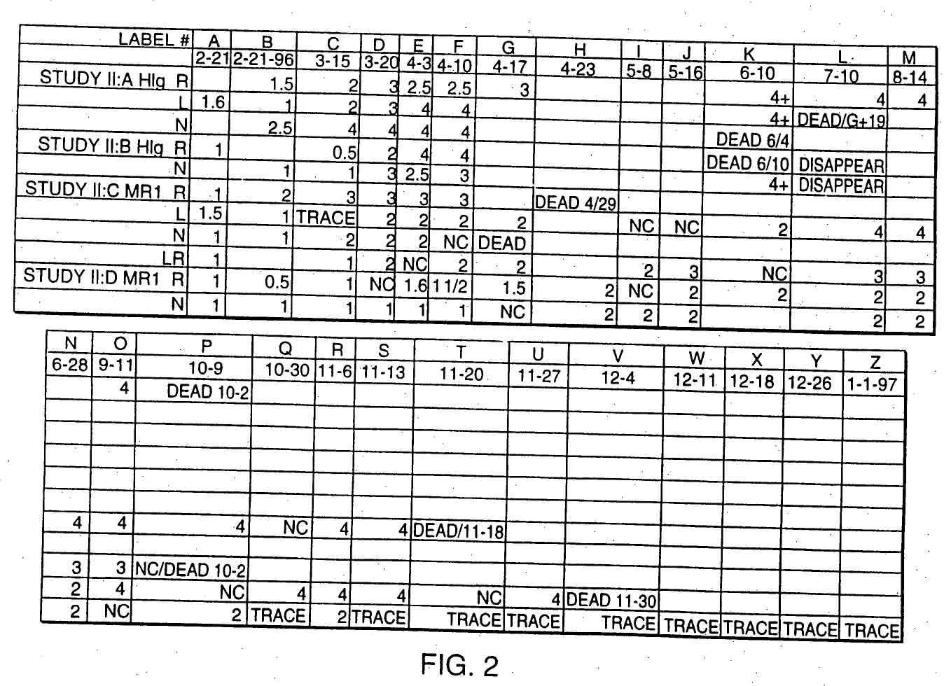

Experiment II: Treatment Begun at 4 Months (FIGS. 1 and 2)

MR1 treatment was initiated when the mice were 4 months of age. MR1 was administered to treated animals once at 500 ug/animal i.p. when the mice were 4 months old, once at 7 months of age, and once at 9 months followed by once-monthly injections. After 4 months of treatment, 4 of the 5 control animals had died, but four of the six treated animals were yet alive. Three of these four previously surviving ted mice died, one each at 12, 13 and 13.5 months. One still survives, and is now 15 months old, an extraordinary longevity for mice of this cross. Of great interest, the surviving animal (mouse II:DN on FIG. 2) had moderate nephritis (2+ proteinuria) from ages 8 to 13 months, which has decreased to only trace levels of proteinuria for the last two months. This is the first demonstration of a functional reversal of nephritis in a mouse of this strain.

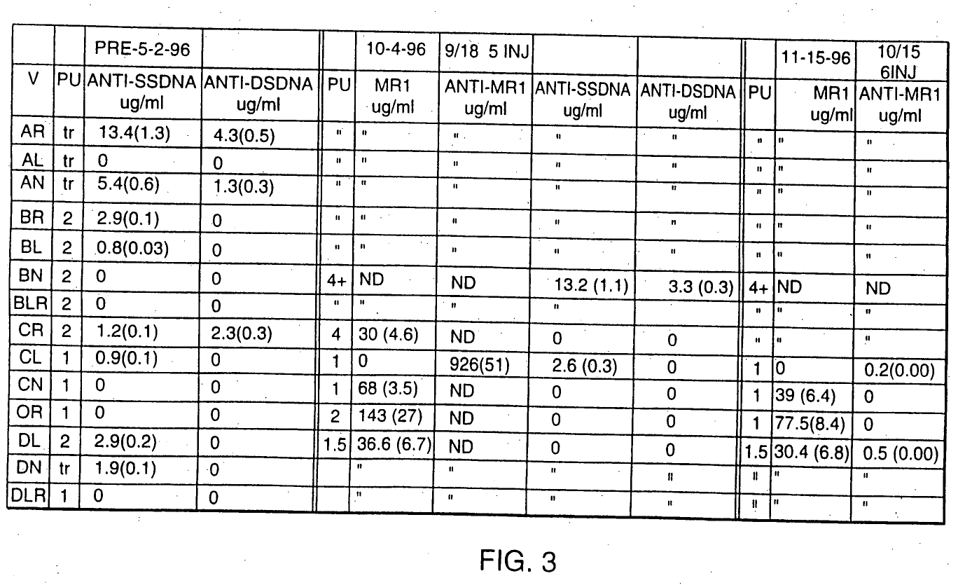

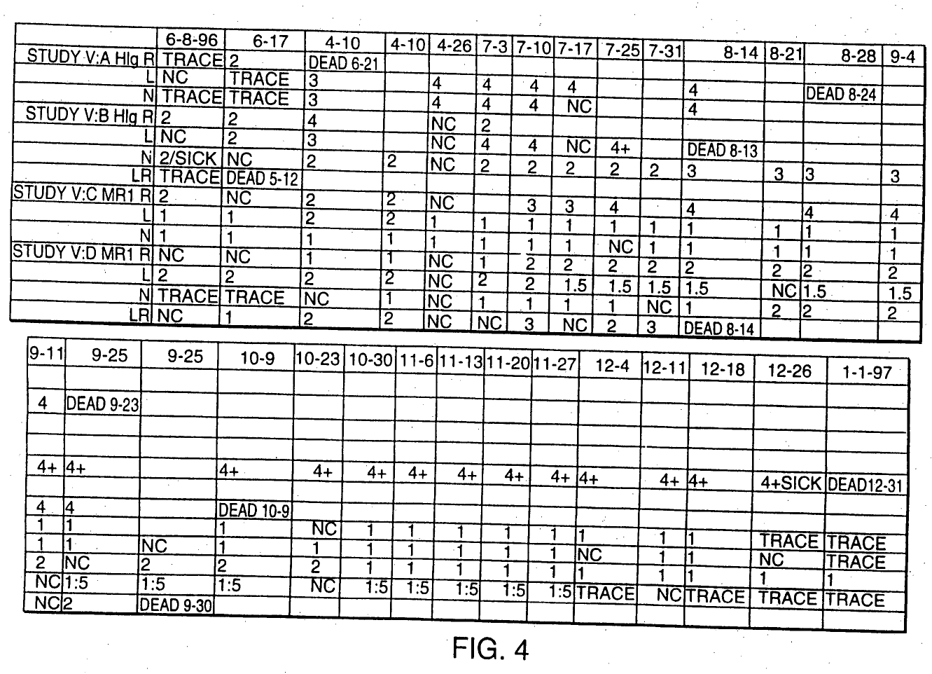

Experiment V: Treatment Begun at 4.5 Months (FIGS. 3 and 4)

MR1 treatment was initiated when the mice were 4.5 months of age. MR1 was administered to treated animals once at 500 ug/animal i.p. when the mice were 4.5 months old, and then as monthly injections of 500 ug, i.p. After 4.5 months, 6 of the 7 control animals had died, but six of the seven treated animals survived. After 8 months of treatment, all controls were dead, but only three of the seven treated mice had died. As shown in FIG. 4, four of the seven MR1-treated animals had their nephritis reversed as shown by sustained lowered proteinuria levels. These four animals are still alive at age 12.5 months.

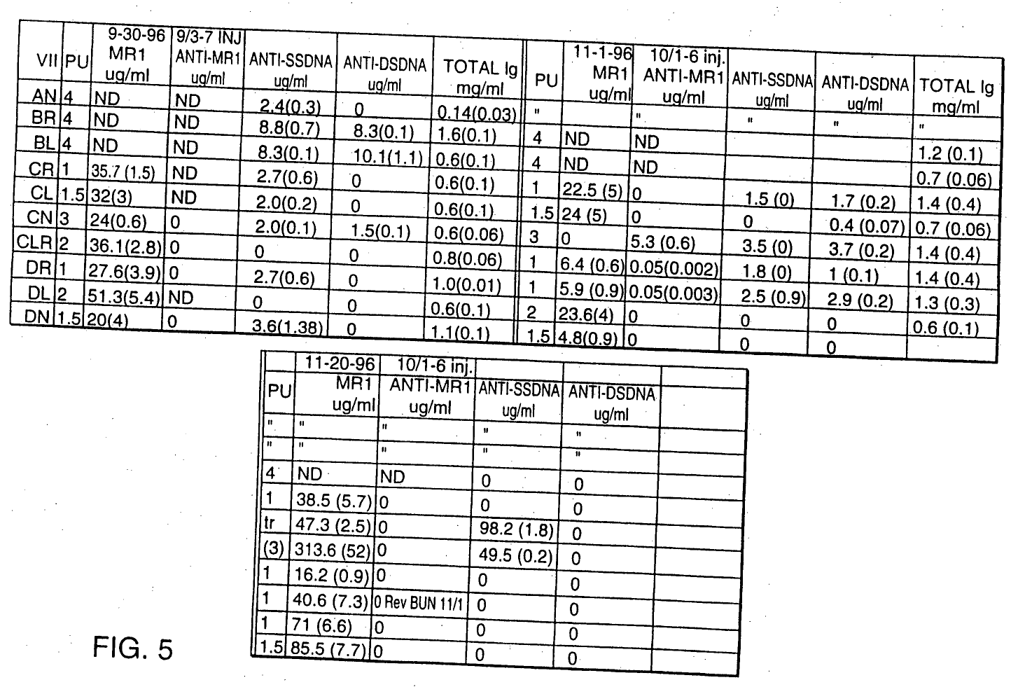

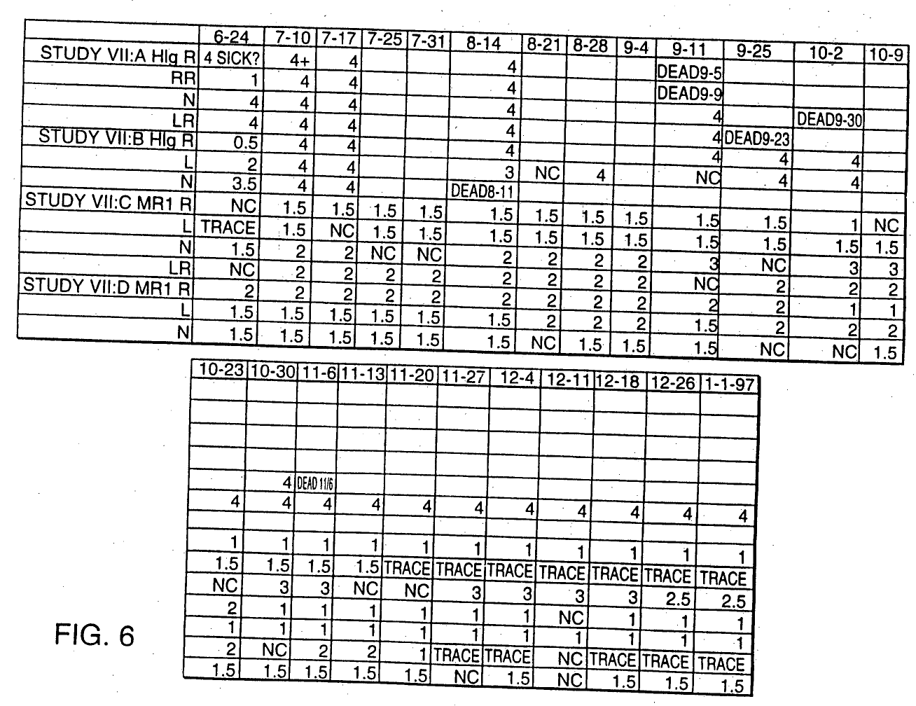

Experiment VII: Treatment Begun at 5.5 Months (FIGS. 5 and 6)

MR1 treatment was the mice were 5.5 months of age. MR1 at 500 μg/animal i.p. was administered to treated animals once weekly for six weeks, followed by monthly injections. After 5 months of treatment, at age 10.5 months, 6 of the 7 control animals had died; all of the 7 treated animals are still alive at age 12 months. The following values were measured in the animals which still survived at 8.5 months, after 3.5 months of treatment:

| | anti-SS-DNA | anti-DS DNA | PU |
|---|---|---|---|
| control | 2.4 | 0 | 4 |
| | 8.8 | 6.3 | 4 |
| | 6.3 | 10.1 | 4 |
| Mean (Std. Dev.) | 5.8 (2.6) | 5.4 (4.1) | 4 (0) |
| MR1 | 2.7 | 0 | 1 |
| | 2.0 | 0 | 1.5 |
| | 2.0 | 1.5 | 3 |
| | 0 | 0 | 2 |
| | 2.7 | 0 | 1 |
| | 0 | 0 | 2 |
| | 3.5 | 0 | 1.5 |
| Mean (Std. Dev.) | 1.8 (1.2) | 0.2 (0.5) | 1.7 ( ) |

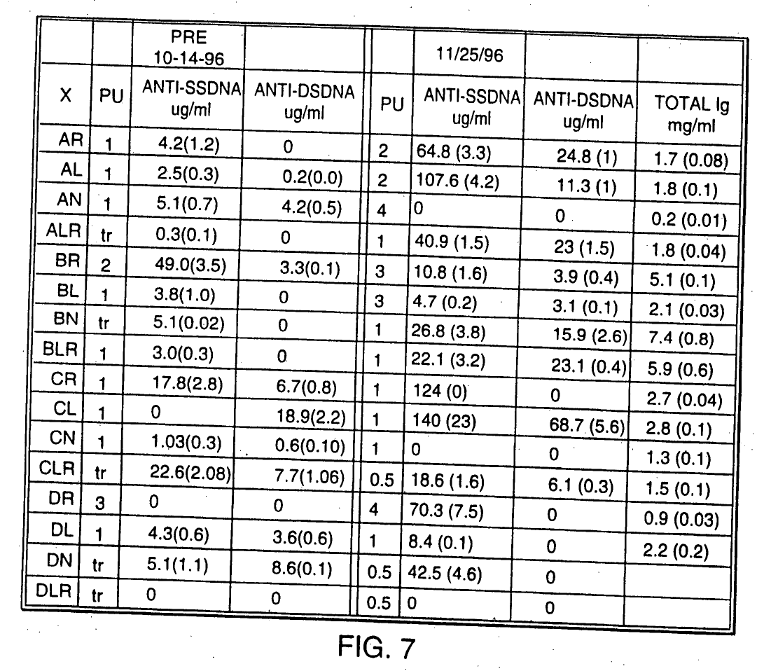

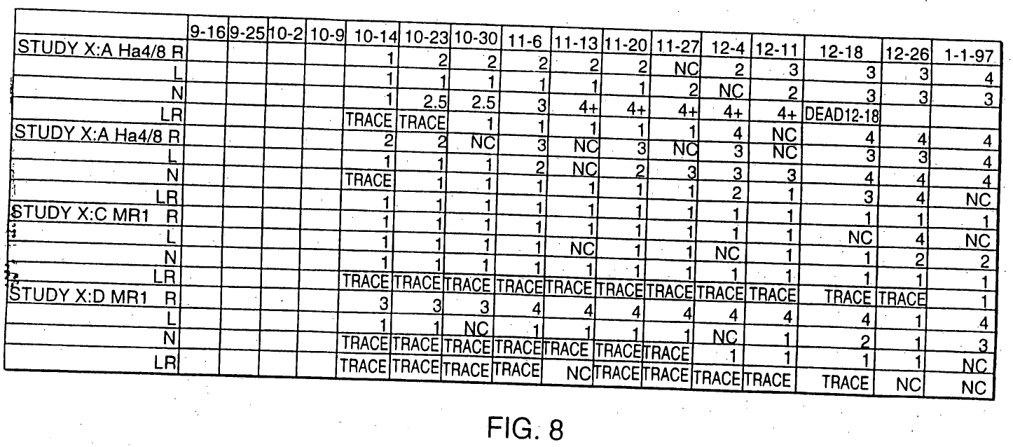

Experiment X: Less Intensive Treatment, Begun at 5.5 Months (FIGS. 7 and 8)

MR1 treatment was initiated when the mice were 5.5 months of age. MR1 was administered to treated animals once weekly at 500 ug/animal i.p. for four weeks, followed by monthly injections of 200 μg, i.p. Of the 16 mice in the study (8 each in control and treated groups), now 8.5 months of age, only one mouse has died, a control animal at age 7.5 months. As shown in FIG. 8, seven of the eight control animals had proteinuria which steadily increased to high levels, averaging +3.4 for the 7 surviving control mice. All but one of the eight MR1-treated mice have maintained low proteinuria, which currently averages +2 for the 8 treated mice. As shown in FIG. 7, six of the treated animals, but only one of the controls, have no detectable anti-dsDNA antibodies.

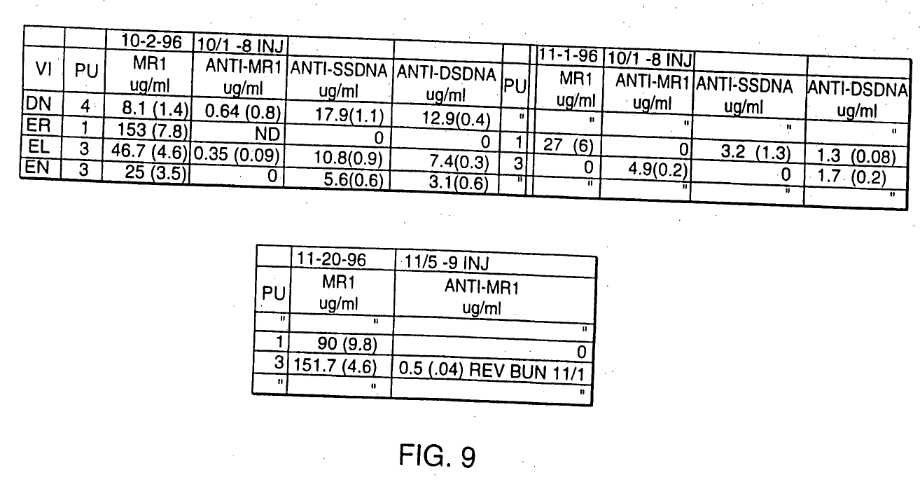

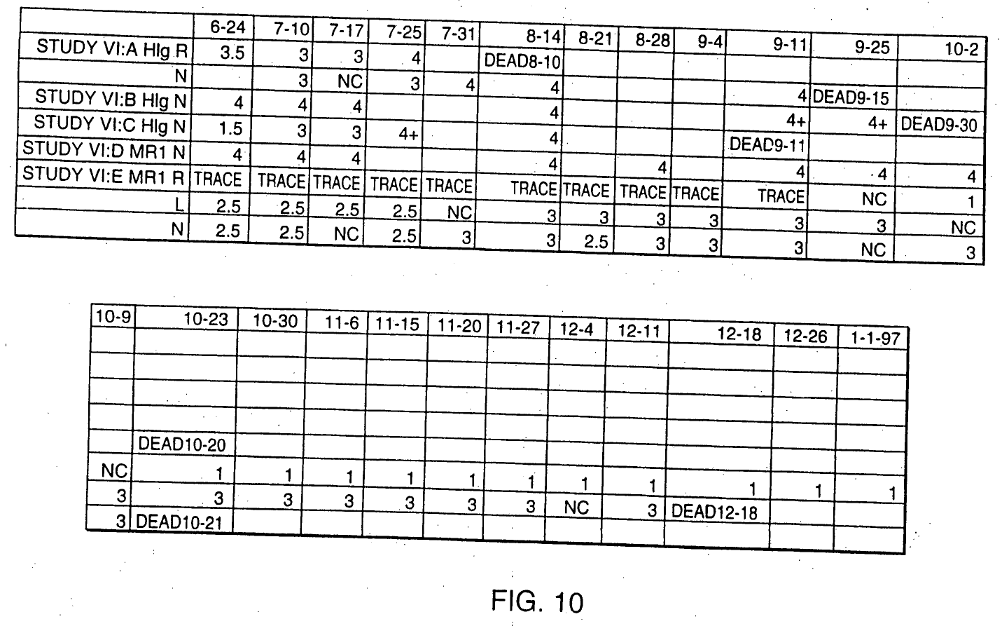

Experiment VI: Treatment Begun at 7 Months (FIGS. 9 and 10)

MR1 treatment was initiated when the mice were seven months of age. MR1 was administered to 4 treated animals once weekly at 500 ug/animal i.p. for six weeks, followed by monthly injections of 500 ug, i.p. By age 10 months, all 4 control animals had died. While 2 of the treated mice died at age 11 months, and a third at 13 months, one of the four treated animals remains alive currently at 14 months of age, after 7 months of treatment. The surviving treated animal (number VI:ER) currently has level 1 proteinuria, and detectable anti-dsDNA and anti-ssDNA antibodies.

These experiments show that treatment of (SWR X NZB) F₁ mice with anti-CD40L mAb, administered for at least a period of time at intervals of over 3 weeks, markedly and consistently prolongs survival as compared to control animals, and slows development of nephritis as indicated by proteinuria levels. In some animals, the treatment actually reverses nephritis, as shown by a reduction in proteinuria levels. Of 32 treated animals, 11 had urine protein levels which decreased with anti-CD40L mAb therapy; none of the control animals had similar reductions. Of 24 treated animals in which serum blood urea nitrogen (BUN) was measured, 3 had decreases in BUN levels after treatment, which was not observed in any control animal. In addition, MR1 treatment often results in a reduced serum concentration of anti-DS and anti-SS DNA autoantibodies, which are normally produced in untreated animals of this type.

The disease-reducing or -preventing results of these experiments demonstrate that anti-CD40L compounds may successfully be used to treat antibody-associated conditions when administered at intervals of 3 or more weeks. This is a surprising and unanticipated finding, which confers significant advantages over previously contemplated dosing regimens. Particularly for patients being treated for a chronic disease, reduced frequency of treatments results in lowered cost, inconvenience, and discomfort, particularly for injectable or intravenous treatments. In addition, any side effects of treatment would be expected to be reduced with fewer and more widely spaced dosings.

Although the foregoing invention has been described in some detail by way of illustration and example for purposes of clarity of understanding, it will be obvious to one skilled in the art that certain changes and modifications may be practiced within the scope of the invention, as limited only by the scope of the appended claims.

1. A method of treating a patient with an antibody-related disease, comprising administering a therapeutically effective amount of an anti-CD40L compound to the patient on a first day and again on a second day, with at least about 3 weeks between the first day and the second day.

2. The method of claim 1, further comprising administering a therapeutically effective amount of an anti-CD40L compound to the patient on a third day, with an interval of at least about 3 weeks between the second day and the third day.

3. The method of claim 1, wherein the interval between the first day and the second day is at least about 4 weeks, at least about 6 weeks, or at least about 8 weeks.

4. A method of treating a patient with an antibody-related disease, comprising administering a therapeutically effective amount of an anti-CD40L compound to the patient for a first therapeutic period at intervals of less than about 3 weeks, then administering a therapeutically effective amount of an anti-CD40L compound to the patient for a second therapeutic period at intervals of at least about 3 weeks.

5. The method of claim 4, wherein the anti-CD40L compound is administered for the second therapeutic period at intervals of at least about 4 weeks.

6. A method of treating a patient with a chronic autoimmune disease, comprising administering a therapeutically effective amount of an anti-CD40L compound to the patient on a first day and again on a second day, with at least about 3 weeks between the first day and the second day.

7. The method of claim 6, further comprising administering a therapeutically effective amount of an anti-CD40L compound to the patient on a third day, with at least about 3 weeks between the second day and the third day.

8. The method of claim 6, wherein there is at least about 4 weeks between the first day and the second day.

9. A method of treating a patient with a chronic immune system disorder, comprising administering a therapeutically effective amount of an anti-CD40L compound to the patient for a first therapeutic period at intervals of less than about 3 weeks, then administering a therapeutically effective amount of an anti-CD40L compound to the patient for a second therapeutic period at intervals of at least about 3 weeks.

10. The method of claim 9, wherein the anti-CD40L compound is administered for the second therapeutic period at intervals of at least about 4 weeks.

11. The method of claim 9, wherein the chronic immune disorder is systemic lupus erythematosus, an allergic disorder, myasthenia gravis, autoimmune hemolytic anemia, idiopathic thrombocytopenic purpura, or anti-phospholipid syndrome.

12. The method of claim 9, wherein the chronic immune disorder is psoriasis, arthritis or multiple sclerosis.

13. The method of claim 9, wherein the anti-CD40L compound is an anti-CD40L antibody.

14. The method of claim 13, wherein the antibody is a monoclonal antibody.

15. The method of claim 14, wherein the monoclonal antibody is 5c8.

16. The method of claim 9, wherein the anti-CD40L compound is formulated in a therapeutic composition comprising a therapeutically-effective amount of the anti-CD40L compound and a pharmaceutically acceptable carrier.

17. The method of claim 16, wherein the therapeutic composition further comprises a second therapeutically effective compound.

18. A method of inhibiting rejection of transplanted tissue within a patient, comprising administering a therapeutically effective amount of an anti-CD40L compound to the patient on a first day and again on a second day, with at least about 3 weeks between the first day and the second day.

19. The method of claim 18, further comprising administering a therapeutically effective amount of an anti-CD40L compound to the patient on a third day, with at least about 3 weeks between the second day and the third day.

20. The method of claim 18, wherein there is at least about 4 weeks between the first day and the second day.

21. The method of claim 18, wherein the transplanted tissue is a kidney, liver, or heart.

22. The method of claim 18, wherein the transplanted tissue is an allograft or a xenograft.

23. A method of inhibiting rejection of transplanted tissue within a patient, comprising administering a therapeutically effective amount of an anti-CD40L compound to the patient for a first therapeutic period at intervals of less than about 3 weeks, then administering a therapeutically effective amount of an anti-CD40L compound to the patient for a second therapeutic period at intervals of at least about 3 weeks.

24. A method of inhibiting immune reaction to the gene product of a transgene within a patient, comprising administering a therapeutically effective amount of an anti-CD40L compound to the patient on a first day and again on a second day, with at least about 3 weeks between the first day and the second day.
